(19) United States
(12) Patent Application Publication
(10) Pub. No.: US 2016/0038155 A1
(43) Pub. Date: Feb. 11, 2016

(54) Glow-In-The-Dark Tourniquet System

(71) Applicant: IPC, Inc., Lakeville, MN (US)

(72) Inventors: Ronald Eric Griffith, Lakeville, MN (US); Martin John Griffith, Lakeville, MN (US)

(73) Assignee: IPC, Inc.

(21) Appl. No.: 14/823,101

(22) Filed: Aug. 11, 2015

Related U.S. Application Data
(60) Provisional application No. 62/035,894, filed on Aug. 11, 2014.

(51) Int. Cl. A61B 17/132

(52) U.S. Cl. CPC A61B 17/1325; A61B 17/1322; A61B 2017/00862

(57) ABSTRACT

A glow-in-the-dark tourniquet system for providing a tourniquet that remains visible in low-light conditions. The glow-in-the-dark tourniquet system generally includes an elongated member having a first surface and a second surface opposite of the first surface adapted to be used as a tourniquet. The elongated member includes a phosphorescent material to visibly glow in low light conditions to prevent the elongated member from being forgotten on a patient. The elongated member also may include one or more indicia on the first surface to provide additional visual information to medical personnel in low light conditions.

### CROSS REFERENCE TO RELATED APPLICATIONS

I hereby claim benefit under Title 35, United States Code, Section 119(e) of U.S. provisional patent application Ser. No. 62/035,894 filed Aug. 11, 2014. The 62/035,894 application is currently pending. The 62/035,894 application is hereby incorporated by reference into this application.

STATEMENT REGARDING FEDERALLY SPONSORED RESEARCH OR DEVELOPMENT

Not applicable to this application.

### BACKGROUND OF THE INVENTION

1. Field of the Invention

The present invention relates generally to tourniquets and more specifically it relates to a glow-in-the-dark tourniquet system for providing a tourniquet that remains visible in low-light conditions.

2. Description of the Related Art

Any discussion of the related art throughout the specification should in no way be considered as an admission that such related art is widely known or forms part of common general knowledge in the field.

Tourniquets have been used for years to restrict venous blood flow to a portion of a body part of a patient such as an arm or leg to allow medical personnel to perform a medical procedure. A tourniquet is typically used to occlude a patient's vein and enable medical personnel to successfully perform a variety of intravenous procedures including, but not limited to, phlebotomy, intravenous catheter insertion, dialysis, blood transfusion, donation, intravenous therapy and infusion set insertion. Conventional tourniquets are typically comprised of non-latex elastic polymers to prevent human latex reactions. Conventional tourniquets also come in various structures such as flat elongated bands or tubing typically around 18 inches in length and 1 inch in width.

When using conventional tourniquets, for example to obtain a blood sample, the medical personnel will normally place the tourniquet about the upper arm of the patient and apply pressure by tightening and slip knotting the tourniquet to induce swelling in the veins in the lower arm. An appropriate venipuncture site is then selected and a needle is inserted to retrieve a blood sample. After the needle is removed, the tourniquet is then removed usually by giving the tourniquet a quick pull to release the slip knot.

Disposable tourniquets are used in low light locations such as intensive care units and accident scenes. Because of the low light conditions combined with the high stress and rushed conditions when using a disposable tourniquet, there is a significant likelihood that healthcare personnel will forget to remove the tourniquet after a medical procedure. If a tourniquet is forgotten on a patient, there is a chance of permanent damage to the body part or death of the patient.

Because of the inherent problems with the related art, there is a need for a new and improved glow-in-the-dark tourniquet system for providing a tourniquet that remains visible in low-light conditions.

### BRIEF SUMMARY OF THE INVENTION

Provided herein is a tourniquet which includes an elongated member having a first surface and a second surface opposite of the first surface adapted to be used as a tourniquet. The elongated member includes a phosphorescent material to visibly glow in low light conditions to prevent the elongated member from being forgotten on a patient. The elongated member also may include one or more indicia on the first surface to provide additional visual information to medical personnel in low light conditions.

An object of the present invention is to provide a glow-in-the-dark tourniquet that remains visible in low light conditions.

Another object of the present invention is to provide a glow-in-the-dark tourniquet that decreases the likelihood of the tourniquet being forgotten on a patient in low light conditions.

A further object of the present invention is to provide a glow-in-the-dark tourniquet that reduces medical errors.

An additional object of the present invention is to provide a glow-in-the-dark tourniquet that prevents patient injuries.

Another object of the present invention is to provide a glow-in-the-dark tourniquet that decreases the likelihood that the tourniquet will visually blend in with the bed coverings or patient gowns in low light conditions.

Another object of the present invention is to provide a glow-in-the-dark tourniquet that decreases the likelihood that the tourniquet will be gathered with the bed coverings or gowns and put through the washing machine which can disintegrate causing problems in the washing equipment.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention comprised of a band structure.

FIG. 2 is a top view thereof.

FIG. 3 is a front view thereof with an indicia.

FIG. 4 is a front view thereof with an alternative indicia.

FIG. 5 is a front view thereof with a second alternative indicia.

FIG. 6 is a front view thereof with a third alternative indicia.

FIG. 7 is a side view of the present invention.

FIG. 8 is an upper perspective view of the present invention attached to the arm of a patient for use as a tourniquet.

FIG. 9 is a cross sectional view of the arm showing the elongated member attached about the arm forming a tourniquet.

FIG. 10 is an upper perspective view of the present invention comprised of a tube structure.

### DETAILED DESCRIPTION OF THE INVENTION

### A. Overview.

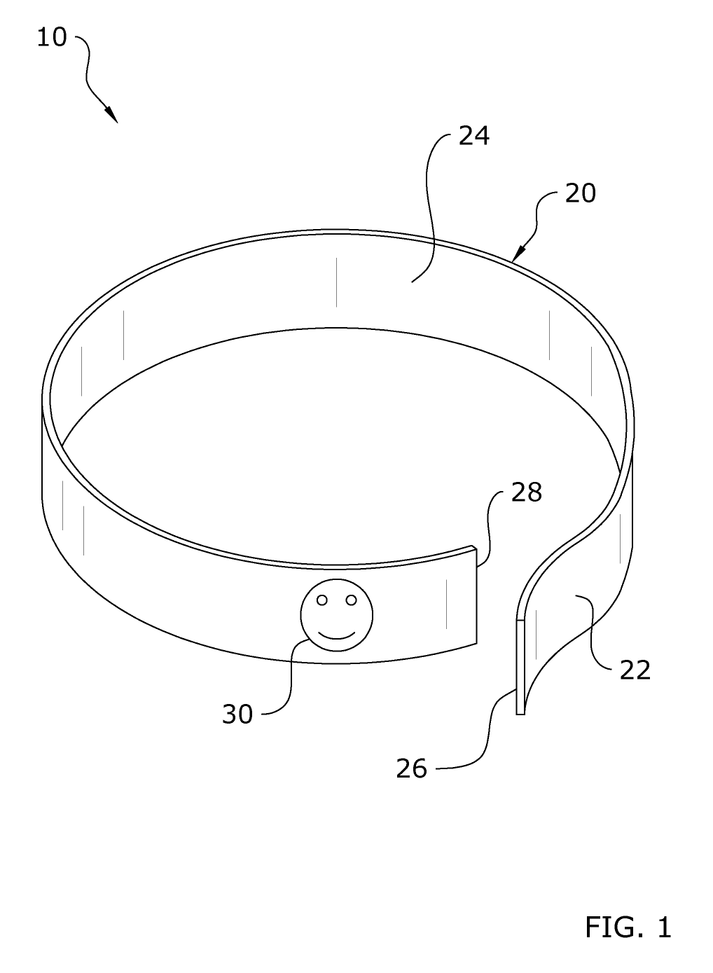

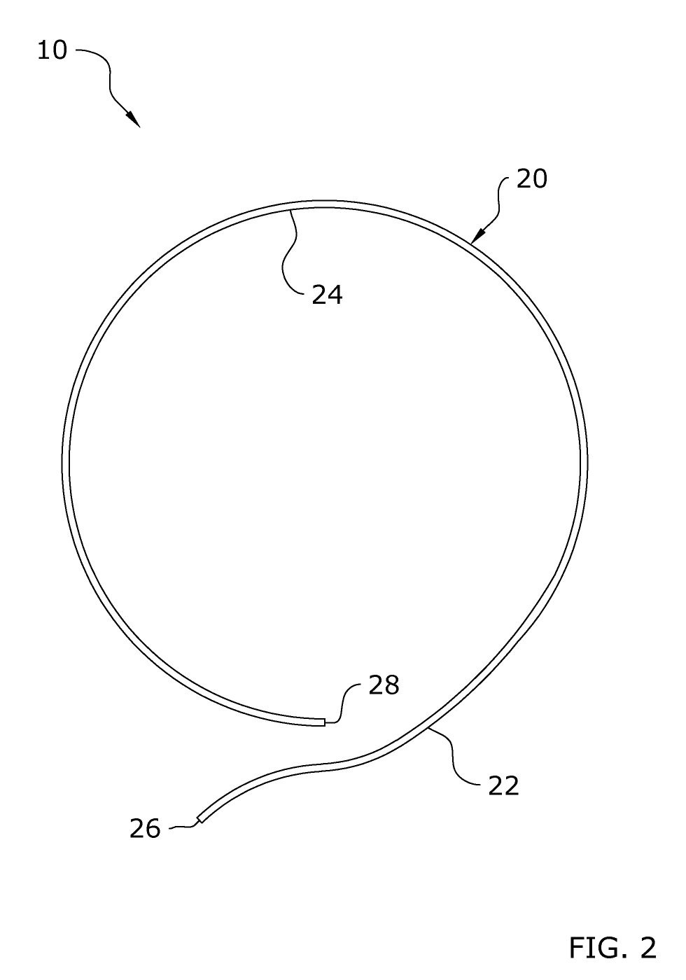

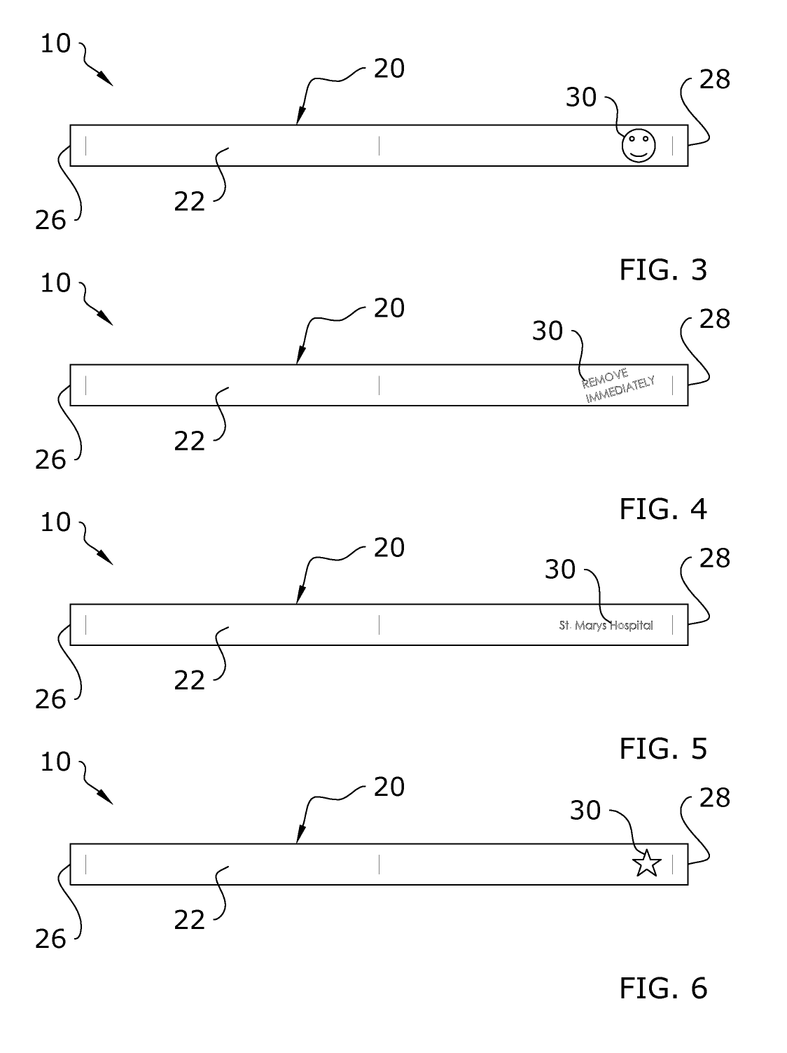

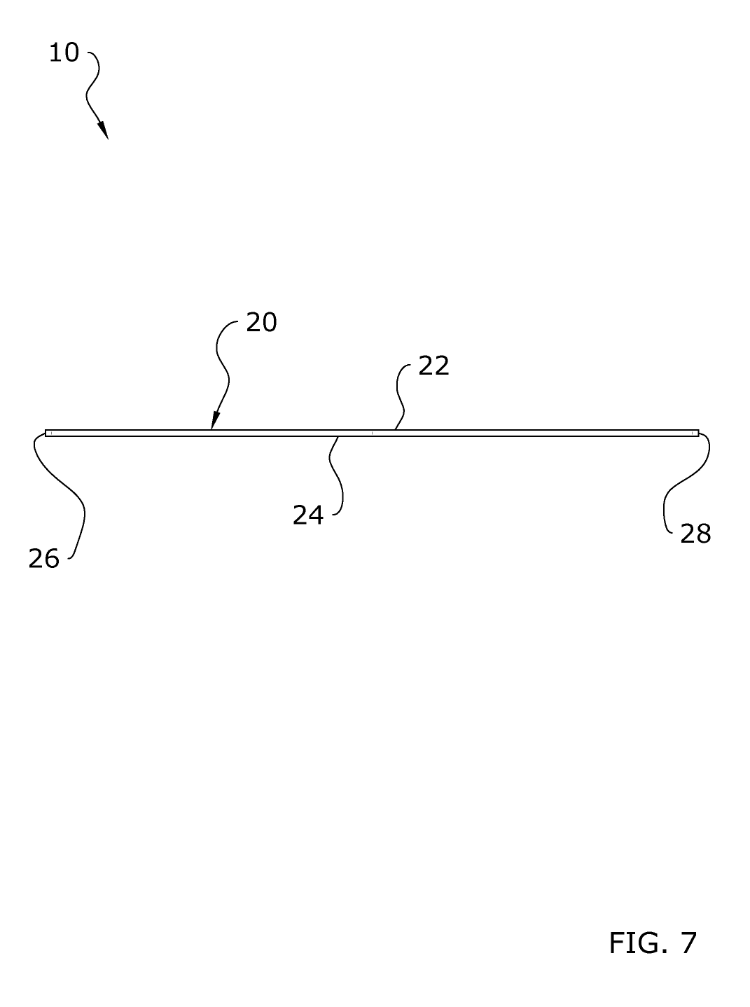

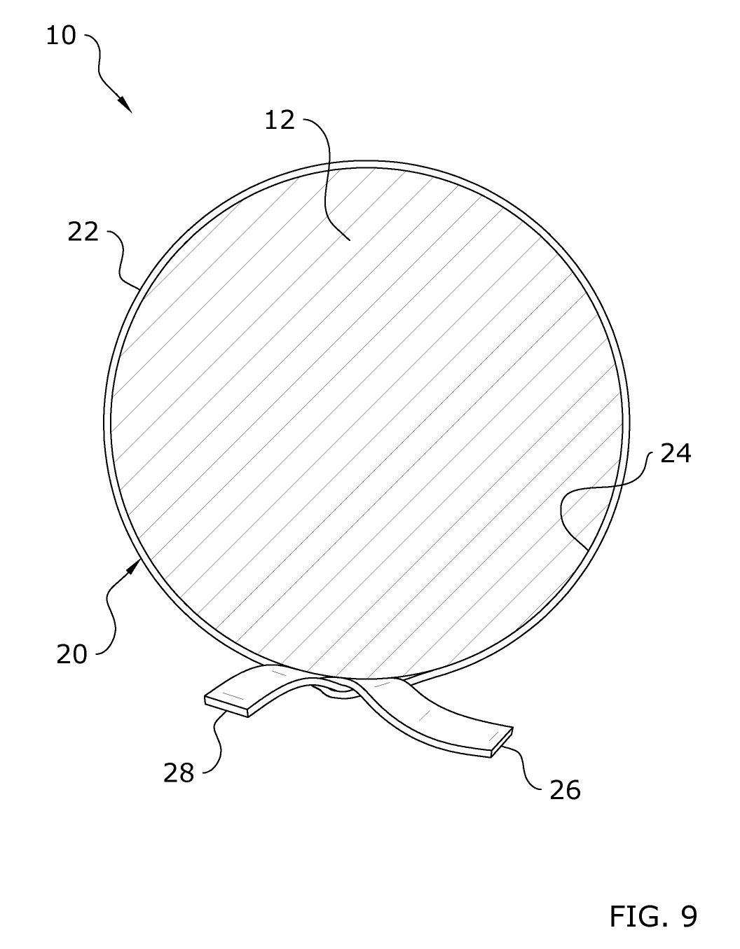

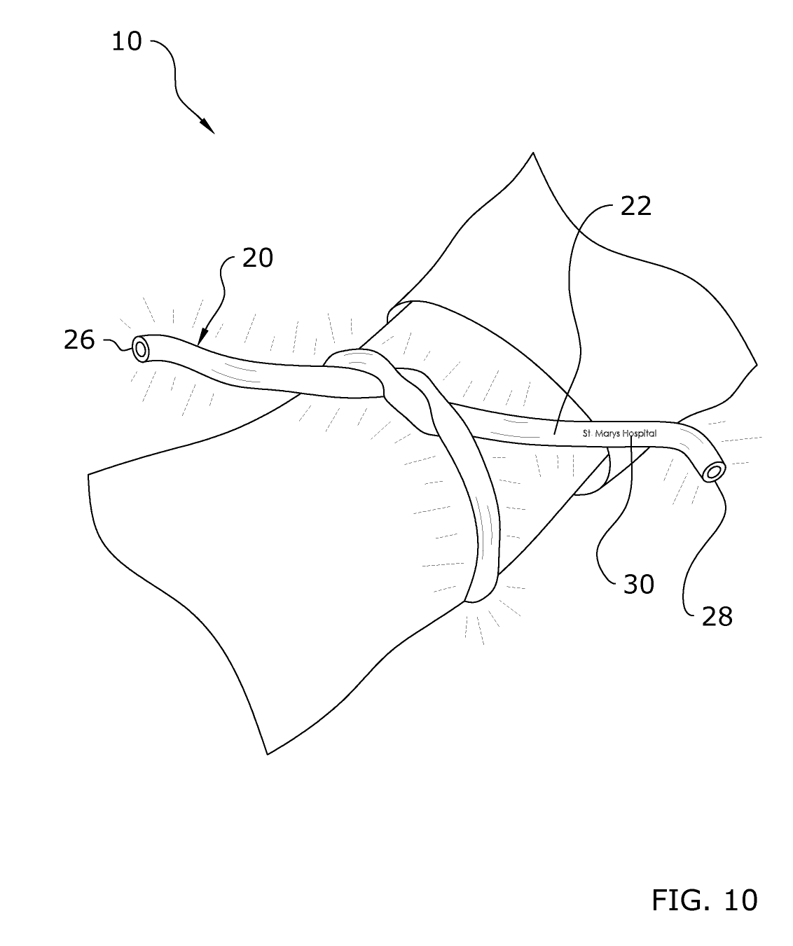

Turning now descriptively to the drawings, in which similar reference characters denote similar elements throughout the several views, FIGS. 1 through 10 illustrate a glow-in-the-dark tourniquet system, which comprises an elongated member 20 having a first surface 22 and a second surface 24 opposite of the first surface 22 adapted to be used as a tourniquet. The elongated member 20 includes a phosphorescent material to visibly glow in low light conditions to prevent the elongated member 20 from being forgotten on a patient. The elongated member 20 also may include one or more indicia 30 on the first surface 22 to provide additional visual information to medical personnel in low light conditions. The tourniquet of the present invention may be used with respect to various types of medical procedures such as, but not limited to, phlebotomy, intravenous catheter insertion, dialysis, blood transfusion, donation, intravenous therapy and infusion set insertion. The invention disclosed is preferably used for medical procedures but may also be used as an exercise band.

The glow-in-the-dark tourniquet is comprised of an elongated strip forming a disposable tourniquet having an outer surface, an inner surface opposite of the outer surface, a first end 26 and a second end 28 opposite of the first end 26. The elongated strip is comprised of a flat structure that has a width, a length and a thickness that may vary. The elongated strip is comprised of a flexible and elastic base material such as a thermoplastic elastomer. The elongated strip includes a colorant (e.g. color masterbatch, colored pellets, colored compounds, colored pigments, etc.) that may be dispersed throughout the entire elongated strip or forming a pattern. A phosphorescent component is added to the elongated strip to provide a visual reminder to medical personnel in reduced lighting conditions to remove the tourniquet (e.g. nighttime, mines, etc.). The phosphorescent component may be comprised of phosphorescent inks, phosphorescent masterbatch, phosphorescent pellets, phosphorescent compounds or phosphorescent pigments. The phosphorescent component may be added throughout the entire elongated strip or only in certain portions of the elongated strip to form patterns such as stripes, symbols and/or words.

### B. Elongated Member.

The elongated member 20 may be comprised of any elongated structure capable of being used as a tourniquet. The elongated member 20 is preferably comprised of an elastic material to allow for resilient stretching of the elongated member 20 similar in function to a conventional disposable tourniquet. However, the elongated member 20 may be comprised of non-elastic materials. At least a portion of the elongated member 20 is phosphorescent (glow-in-the-dark) to provide visibility of at least a portion of the elongated member 20 when attached to a patient as a tourniquet in low light conditions (e.g. darkness, dim light). The elongated member 20 may include phosphorescent material within the composition of the elongated member 20 and/or phosphorescent material applied to an exterior surface of the elongated member 20. The elongated member 20 is preferably comprised of a disposable material.

The elongated member 20 is preferably comprised of an elongated flat elastic band as illustrated in FIGS. 1 through 9 of the drawings. Alternatively, the elongated member 20 may be comprised of other structures such as, but not limited to, elongated elastic tubes as illustrated in FIG. 10 of the drawings. The width, thickness and length of the elongated flat elastic band may vary depending upon the desired medical usage (e.g. 1 inch width, ⅛ inch thickness and 18 inches length). Various other widths, thicknesses and lengths may be used for the elongated member 20. The elongated member 20 may also have a wide variety of physical properties such as, but not limited to, soft, stretchy, elastic and anti-slip.

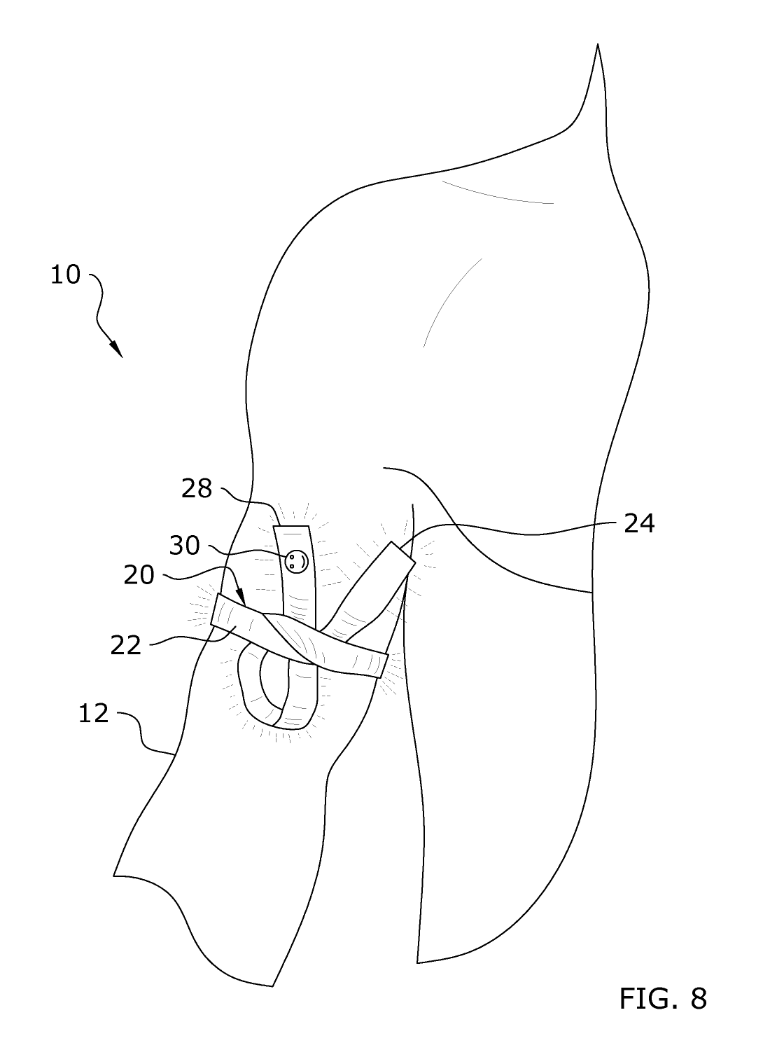

Regardless of the structure of the elongated member 20, the elongated member 20 preferably includes a first end 26 and a second end 28 opposite of the first end 26 as illustrated in FIGS. 1 through 7 of the drawings. The elongated member 20 further includes a first surface 22 (e.g. exterior surface) and a second surface 24 (e.g. interior surface) opposite of the first surface 22. The second surface 24 is the surface that contacts the skin of the patient with a portion of the first surface 22 contacting the skin of the patient also as illustrated in FIG. 8. The first surface 22 is preferably an exterior surface of the elongated member 20 wherein a significant portion or all of the first surface 22 is visible when the elongated member 20 is attached to a body part of a patient as a tourniquet. The first surface 22 and/or the second surface 24 may be smooth or textured.

The elongated member 20 preferably has side edges that are of equal length and are spaced apart consistently that extend from the first end 26 to the second end 28. The elongated structure may also have notched sides or internal cutouts that provide a functional method of securing the tourniquet while in use. The elongated member 20 may also include internal cutouts that form a pattern such as a symbol, shape or word.

### C. Composition of Elongated Member.

1. Base Material.

The elongated member 20 is preferably comprised of an elastic material, but the elongated member 20 may be comprised of a non-elastic material. The elongated member 20 may be comprised of any material that exhibits properties similar to natural rubber or latex. The elongated member 20 is preferably comprised of a polymer such as, but not limited to, thermoplastic elastomers and thermoplastic polymers (e.g. polypropylene). Examples of suitable thermoplastic elastomers include, but are not limited to, polyolefin blends, dynamically vulcanized blends, copolyesters, styrenics, styrenic/polyolefin blends, nylons, polyurethanes and metallocene polymers.

2. Colorant.

The elongated member 20 further preferably includes a colorant added to the base material to provide any desired color (e.g. white, violet, blue, green, yellow, red, orange, pink). One or more colors may be used within the elongated member 20 (e.g. one color for the main body and another color for the indicia 30). The color may be evenly dispersed throughout the elongated member 20 or the color may be used in the elongated member 20 to form a pattern such as symbols, shapes, stripes, characters or words.

Examples of suitable colorants include, but are not limited to, color masterbatch, colored pellets, colored compounds, colored pigments. The colorants may be a masterbatch colorants that is a solid or a liquid additive used for coloring polymers (color masterbatch) such as plastics or imparting other properties to plastics (additive masterbatch). Instead of a colorant added to the base material, the colorant may be printed on the surface 22, 24 of the elongated member 20 such as by using colored inks.

3. Phosphorescent Material.

The elongated member 20 includes a phosphorescent material (i.e. glow-in-the-dark material) either integrally formed within the elongated member 20 during manufacture and/or attached to the surface 22, 24 of the elongated member 20 as a layer of phosphorescent material. The phosphorescent material may be evenly distributed throughout the elongated member 20 (e.g. completely through the entire elongated member 20) or the phosphorescent material may be used in the elongated member 20 to form a visual indicia 301 such as symbols, shapes, patterns, stripes, characters or words. The phosphorescent material is any material that shows an afterglow in darkened light conditions after being charged by a light source (e.g. sun, building lights). Typically, phosphorescent materials continue to emit an afterglow several hours after exposure to the light source has ceased.

The elongated member 20 may include any type of phosphorescent (i.e. glow-in-the-dark) component that is added to and mixed with the base material and the colorant of the elongated member 20 such as, but not limited to, phosphorescent masterbatch (e.g. polymer additive), additive masterbatch, phosphorescent pellets, phosphorescent compounds and phosphorescent pigments. The phosphorescent component/material is added to the base material during manufacturing and production of the elongated member 20. Examples of suitable phosphorescent materials include, but are not limited to, phosphorescent pigments based on zinc sulfide (ZnS), radioisotopes or strontium oxide aluminate. The phosphorescent material may be comprised of any color (lighted and afterglow) such as, but not limited to, white, violet, blue, green, yellow, red, orange, pink.

The phosphorescent material may also be printed on the surfaces 22, 24 of the elongated member 20 such as by using a phosphorescent ink. The printing of the phosphorescent material may be evenly layered throughout the surface of the elongated member 20 or in a manner that forms symbols, characters, one or more words, shapes, stripes, patterns within the surface of the elongated member 20.

### D. Indicia.

The elongated member 20 may also include one or more indicia 30 to provide additional visual information to medical personnel. The indicia 30 may be comprised of any structure, shape or configuration that provides visual information to medical personnel. Examples of suitable indicia 30 include, but are not limited to, symbols, characters, one or more words, shapes, stripes, patterns, etc. FIGS. 1, 3-6, 8, 10 provide examples of indicia 30 such as a smiley face, a star, the words “REMOVE IMMEDIATELY” and the name of a hospital (e.g. “St. Mary's Hospital”). The indicia 30 is preferably part of the first surface 22 but may also be part of the second surface 24. The indicia 30 may also be part of the edges of the elongated member 20.

The indicia 30 may be printed on a surface 22, 24 of the elongated member 20 using conventional printing processes or embedded in the surface of the elongated member 20. The indicia 30 may also embedded throughout the entire thickness of the elongated member 20 (i.e. from the first surface 22 to the second surface 24). The indicia 30 may be formed using the phosphorescent material either printed on the surface or embedded within the elongated member 20 during manufacturing. The indicia 30 may also be comprised of non-phosphorescent material thereby being visually displayed as a darkened area on the first surface 22 of the elongated member 20.

The indicia 30 may be comprised of any color such as, but not limited to, white, violet, blue, green, yellow, red, orange, pink. It is preferable that the color of the indicia 30 is different from the color of the phosphorescent material. The indicia 30 may have more than one color and may have a dark (e.g. black) border).

### E. Operation of Preferred Embodiment.

In use, elongated member 20 is exposed to a light source (e.g. sun, building lights) for a period of time wherein the phosphorescent material absorbs the light and is therefore charged. The exposure to the light source may occur prior to or during the medical procedure.

The user places the disposable elongated member 20 about the upper arm 12 of the patient and applies pressure by tightening and slip knotting the tourniquet to induce swelling in the veins in the lower arm 12 as illustrated in FIG. 8 of the drawings. After the medical procedure is performed, the tourniquet is removed by giving the tourniquet a quick pull to release the slip knot. The tourniquet is then disposed of in accordance with normal and safe procedures.

The phosphorescent material in the elongated member 20 will show an afterglow in low light conditions typically for several hours after exposure to the light source has ceased. In the event that a user forgets to remove the tourniquet from the patient's arm 12, the afterglow of the phosphorescent material will provide a visual reminder to medical personnel in low light conditions to remove the and dispose of the tourniquet. In lighted conditions, the colorant used for the elongated member 20 will provide a visual reminder to medical personnel to remove and dispose of the tourniquet.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A glow-in-the-dark tourniquet, comprising:
an elongated member having a first surface and a second surface opposite of said first surface adapted to be used as a tourniquet;
wherein said elongated member includes a first end and a second end opposite of said first end;
wherein said elongated member is at least partially phosphorescent to glow in low light conditions when said elongated member is attached to a body part of a patient during a medical procedure to visually indicate to medical personnel that said elongated member is attached as a tourniquet on said body part to prevent said elongated member from being forgotten.

2. The glow-in-the-dark tourniquet of claim 1, wherein said elongated member is completely phosphorescent.

3. The glow-in-the-dark tourniquet of claim 1, wherein said elongated member is comprised of a base material, a colorant material and a phosphorescent material.

4. The glow-in-the-dark tourniquet of claim 3, wherein said phosphorescent material is attached as a layer to said first surface of said elongated member.

5. The glow-in-the-dark tourniquet of claim 4, wherein said phosphorescent material is comprised of an ink.

6. The glow-in-the-dark tourniquet of claim 3, wherein said phosphorescent material is mixed with said base material and said colorant material.

7. The glow-in-the-dark tourniquet of claim 6, wherein said phosphorescent material is comprised of an additive masterbatch.

8. The glow-in-the-dark tourniquet of claim 6, wherein said phosphorescent material is comprised of a phosphorescent pigment having a first color.

9. The glow-in-the-dark tourniquet of claim 8, including an indicia on said first surface of said elongated member, wherein said indicia has a second color that is different from said first color.

10. The glow-in-the-dark tourniquet of claim 1, wherein said elongated member is comprised of a polymer having elastic properties.

11. A glow-in-the-dark tourniquet, comprising:
an elongated member having a first surface and a second surface opposite of said first surface adapted to be used as a tourniquet;
wherein said elongated member is comprised of a polymer having elastic properties;
wherein said elongated member includes a first end and a second end opposite of said first end;
wherein said elongated member is at least partially phosphorescent to glow in low light conditions when said elongated member is attached to a body part of a patient during a medical procedure to visually indicate to medical personnel that said elongated member is attached as a tourniquet on said body part to prevent said elongated member from being forgotten; and
an indicia exposed upon said first surface of said elongated member, wherein said indicia is comprised of a symbol, pattern or one or more words.

12. The glow-in-the-dark tourniquet of claim 11, wherein said elongated member is completely phosphorescent.

13. The glow-in-the-dark tourniquet of claim 11, wherein said elongated member is comprised of a base material, a colorant material and a phosphorescent material.

14. The glow-in-the-dark tourniquet of claim 13, wherein said phosphorescent material is attached as a layer to said first surface of said elongated member.

15. The glow-in-the-dark tourniquet of claim 14, wherein said phosphorescent material is comprised of an ink.

16. The glow-in-the-dark tourniquet of claim 13, wherein said phosphorescent material is mixed with said base material and said colorant material.

17. The glow-in-the-dark tourniquet of claim 16, wherein said phosphorescent material is comprised of an additive masterbatch.

18. The glow-in-the-dark tourniquet of claim 16, wherein said phosphorescent material is comprised of a phosphorescent material having a first color.

19. The glow-in-the-dark tourniquet of claim 18, wherein said indicia is comprised of a phosphorescent material having a second color, wherein said second color is different than said first color.

20. A glow-in-the-dark tourniquet, comprising:
an elongated member having a first surface and a second surface opposite of said first surface adapted to be used as a tourniquet;
wherein said elongated member is comprised of a polymer having elastic properties;
wherein said elongated member includes a first end and a second end opposite of said first end;
wherein said elongated member is at least partially phosphorescent to glow in low light conditions when said elongated member is attached to a body part of a patient during a medical procedure to visually indicate to medical personnel that said elongated member is attached as a tourniquet on said body part to prevent said elongated member from being forgotten; and
an indicia exposed upon said first surface of said elongated member, wherein said indicia is comprised of a visual indicator that visually communicates information to said medical personnel;
wherein said elongated member is comprised of a base material, a colorant material and a phosphorescent material, wherein said phosphorescent material is comprised of an additive masterbatch and wherein said phosphorescent material is comprised of a phosphorescent material having a first color;
wherein said indicia is comprised of a phosphorescent material having a second color, wherein said second color is different than said first color.
